(19) United States
(12) Patent Application Publication
(10) Pub. No.: US 2023/0166024 A1
(43) Pub. Date: Jun. 1, 2023

(54) PORTABLE RACK FOR INFUSION DEVICES

(71) Applicant: Fresenius Vial SAS, Brézins (FR)

(72) Inventors: Frederic Couillaud, Coublevie (FR); Gregory Daubelcourt, Brézins (FR); Mathieu Paoli, La Murette (FR); Philippe Traversaz, Voiron (FR)

(21) Appl. No.: 17/997,095

(22) PCT Filed: May 19, 2021
(86) PCT No.: PCT/EP2021/063368

§ 371 Date: Oct. 25, 2022

(30) Foreign Application Priority Data
May 20, 2020 (EP) 20290044

(51) Int. Cl. A61M 5/14

(52) U.S. Cl. CPC A61M 5/1415; A61M 2209/082

(57) ABSTRACT

The invention relates to portable rack (20) for holding infusion devices (2) for administering a medical fluid to a patient, comprising: a housing (21) with a multiplicity of slots (100) wherein the infusion devices (2) can be attached to said multiplicity of slots (100), a clamp (27) for mounting the rack (20) to a pole and/or a rail, and a handle (22) for manually transporting the rack (20).

The invention relates to a portable rack for holding infusion devices for administering a medical fluid to a patient according to claim 1.

An infusion station comprises a rack and a control device, the rack defining a multiplicity of slots for receiving a multiplicity of infusion devices. A system of this kind may generally be used at the bedside of a patient in a healthcare environment, for example within an intensive care unit (ICU) of a hospital. Using an infusion station of the type described herein a multiplicity of infusion devices, such as syringe infusion pumps or volumetric (peristaltic) infusion pumps, may be combined in order to administer one or multiple medical fluids, such as medications for the treatment of the patient or nutritional solutions for the enteral or parenteral feeding of the patient, to a patient in a concerted fashion.

Furthermore, using the infusion station infusion devices may be organized and physically held to form for example a stack of infusion devices at the bedside of the patient, such that an organized and easy-to-handle arrangement of infusion devices is obtained. The rack herein serves as a communication link in that it links infusion devices to each other and to the control device, the rack hence serving as a communication spine which in addition may provide for a power supply for electrically feeding the infusion devices connected thereto.

When the patient is displaced into different wards or services depending on his condition and the medical examinations, the infusion devices need to move with the patient as well.

For this purpose, either the entire infusion station is moving together with the patient or the infusion device is removed from the infusion station and for instance carried on the bed of the patient to move together with the patient. After the removal from the infusion station, the infusion devices are running on battery only. However, such modules are not intended to run on battery for extended lengths of time. For recharging the battery, the infusion device needs to be re-inserted in a rack of the infusion station. However, such a rack is not everywhere available in a hospital and can be cumbersome to move with the patient.

It is an object of the present invention to provide a reliable and durable power supply to an infusion device, even after the removal from the rack of an infusion station.

It is also an object of the invention to provide an infusion pump support that can be handled and moved by an individual user.

These objects are achieved by means of the portable rack comprising the features of claim 1. Various embodiments of the invention are subject of the dependent claims, of the description and of the drawings.

The invention is described by a portable rack for holding infusion devices for administering a medical fluid to a patient, comprising: a housing with a multiplicity of slots wherein infusion devices can be attached to said multiplicity of slots, a clamp for mounting the portable rack to or on a pole and/or a rail, and a handle for manually transporting the portable rack and preferably the infusion devices attached to the rack. The portable rack is a rack for holding and transporting the infusion devices.

In one embodiment the portable rack is characterized in that the portable rack provides power supply to the infusion devices and a communication link for the infusion devices.

In a further embodiment the portable rack is characterized in that the portable rack provides power supply to the infusion devices only. The portable rack provides no communication link. Modules are not intended to run on battery for extended lengths of time. However, they maintain their full performance when running on battery. The portable rack is intended to act as a support mechanism and centralized power supply for several, preferably two, infusion modules, in particular when the preferably two infusion modules are running on battery and need to be transported. The portable rack preferably has no communication capabilities and does not influence the alarms of the infusion modules. The portable rack does not have an embedded software.

In a further embodiment the portable rack is characterized in that the portable rack provides two slots for attaching two infusion devices only. The portable rack is designed to hold and power two modules: a volumetric pump or a syringe pump. For instance, one volumetric pump and one syringe pump, two volumetric pumps or two syringe pumps could be held and powered by the portable rack. With this device, the modules can be carried with the handle or the modules can be attached to a pole or rail. The portable rack with two modules inserted has a weight which still can be carried manually by a user.

To support the handling of the portable rack, preferably the handle is arranged on the top side of the portable rack and is preferably integral with the housing of the portable rack. In one embodiment the handle is provided by an opening in the top side of the housing.

In one embodiment the handle is positioned in the portable rack such that, when at least one infusion device is loaded onto a slot, the back of the portable rack stands essentially vertically, during manual transportation of the portable rack. Essential vertical means that portable rack could be slightly tilted, in particular during transportation.

Preferably, the handle is provided in the front part of the top side of the housing. In this way, the handle is positioned such that, when at least one infusion device is loaded onto a slot, the back of the portable rack stands vertically during manual transportation of the portable rack. In consequence, during manual transportation, the screens of the pumps can still be seen. I.e. the slots of the portable rack are directed in an upright or upper direction during manual transportation and preferably the screens of the pumps can be seen during transportation.

Preferably the clamp is a pole clamp. In one embodiment the clamp is a screw pole clamp. This enables an easy mounting of the portable rack to a pole and/or a rail. To speed up the mounting of the portable rack to the pole, the clamp preferably has a release mechanism, preferably a release button, for sliding the screw pole clamp forward and for tighten it. In particular, the release mechanism is activated by the release button. Also, the orientation of the clamp could be changed for instance by rotation. In this embodiment the portable rack is characterized in that the clamp can be positioned vertically and horizontally.

In a further embodiment the portable rack is further characterized in that the portable rack can be placed or set down directly onto an essentially horizontal and/or preferably flat surface. Preferably the portable rack has a stable and/or flat base. In one embodiment the portable rack has rubber feet. This is useful in situations where no pole is available.

The infusion system is intended for a single patient at a time. The components, but not the disposables, can be reused on multiple patients throughout their lifetime. The portable rack is intended for use by qualified and trained healthcare professionals, including but not limited to the following: nurses (primary users), physicians, nurse practitioners and physician assistants.

The portable rack is intended for use in the following clinical healthcare facilities:
Trauma resuscitation room
Adult Intensive Care Unit
Pediatric and Neonatal Intensive Care Unit
Operating suite
Emergency services
Medicine (general or specialized), surgery and obstetrics
Ambulatory Care Unit in hospital
In-between wards

The idea underlying the invention shall subsequently be described in more detail with reference to the embodiments shown in the figures. Herein:

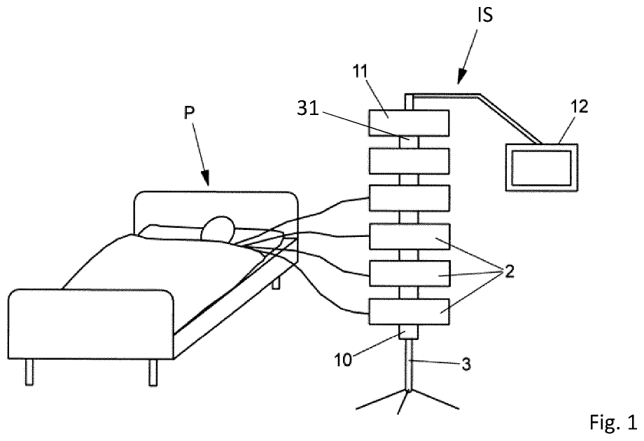

FIG. 1 shows a schematic drawing of an infusion station at the bedside of a patient;

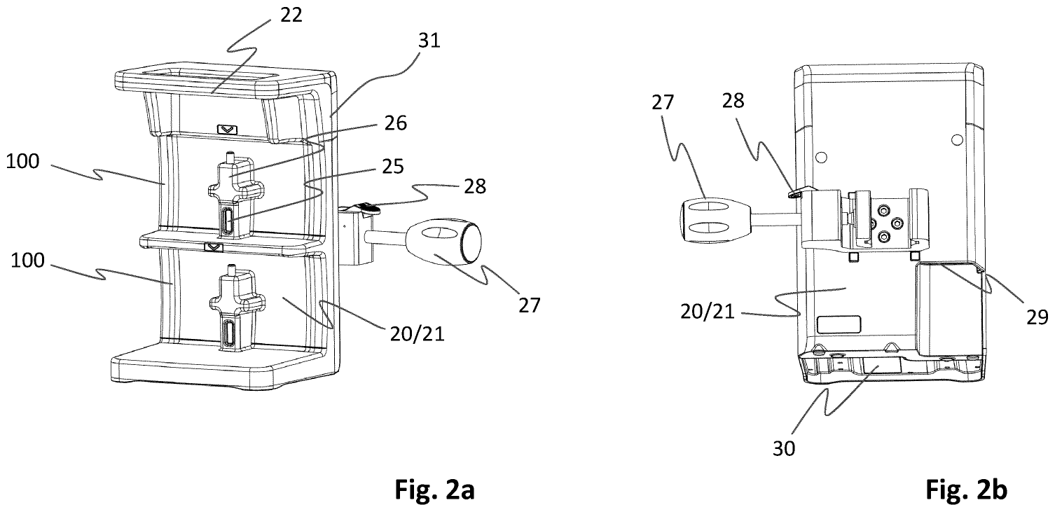

FIG. 2a-b show a front view (FIG. 2a) and a back view of an embodiment of the portable rack;

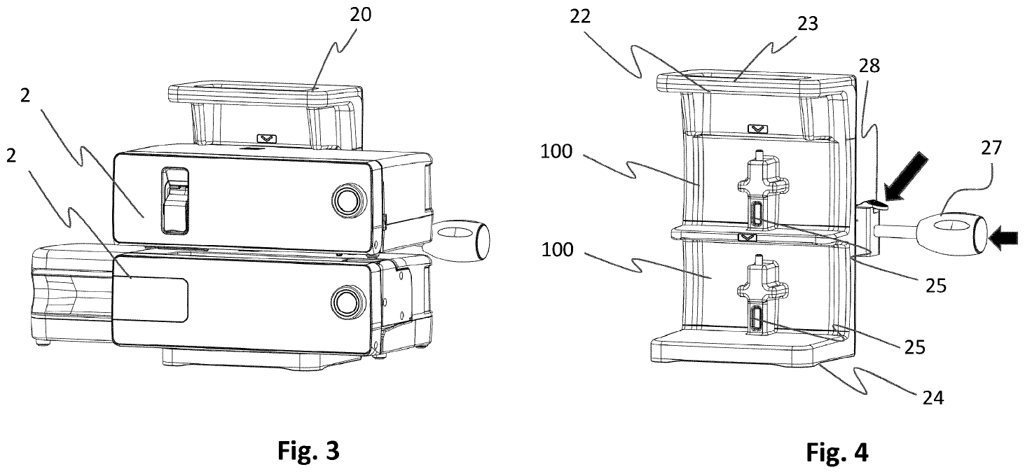

FIG. 3 shows the portable rack of FIGS. 2a and 2b with inserted infusion devices;

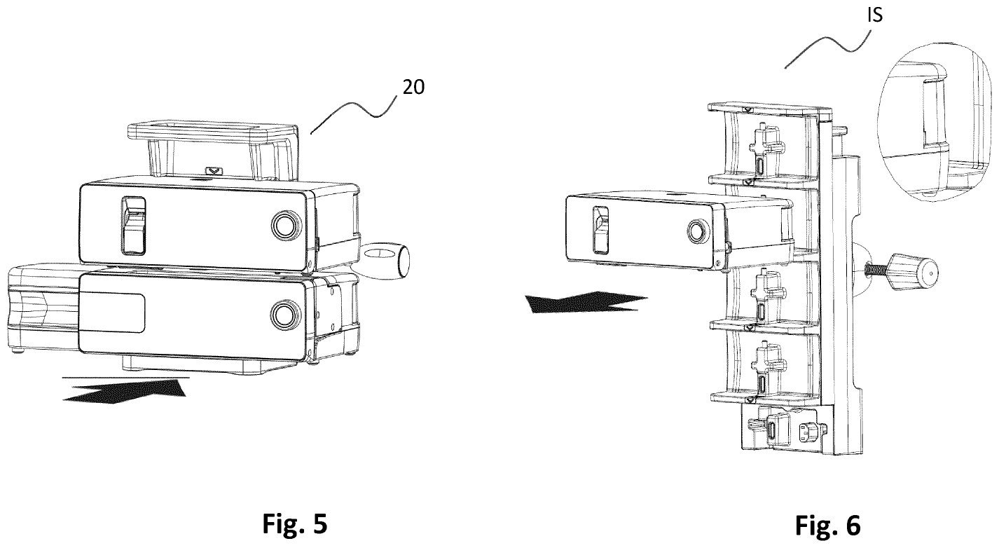

FIG. 4-5 illustrate installing the portable rack and the modules;

FIG. 6-8 illustrate removal and transport of the portable rack and the modules.

FIG. 9 shows the portable rack with two inserted infusion devices;

As an introduction, FIG. 1 shows a schematic drawing of an infusion station IS at the bedside of a patient P, for example in a healthcare environment, such as in an intensive care unit (ICU) of a hospital.

As visible from FIG. 1 and in view of the following figures, the infusion station IS comprises a rack 10 defining a multiplicity of slots 100 to which a multiplicity of infusion devices 2 may be attached. The rack 10, at the slots 100, provides for a mechanical and electrical connection for the infusion devices 2, the rack 10 generally serving as a communication link as well as a power supply such that the infusion devices 2 connected to the rack 10 are interconnected with each other and further are connected to a power supply via the rack 10. As an example, the rack 10 itself does not comprises control device 11. But a control device 11 could be attached to the rack 10 as shown in FIG. 1. The rack 10 stands vertically and the slots 100 extends perpendicularly to the back 31 of the rack 10 such that the infusion devices are horizontally positioned onto the rack. As a consequence, the screen of the infusion devices 2 can be easily seen by a user.

The rack 10 and the infusion devices 2 arranged thereon are connected to a stand 3 placed at the bedside of a patient P, as visible from FIG. 1. The rack 10 holds the infusion devices 2 connected thereto in an organized fashion, forming a vertical stack of infusion devices 2.

The infusion devices 2 may be configured as syringe infusion pumps or as volumetric (peristaltic) infusion pumps. In either case, the infusion devices 2 may comprise a housing, an input device for example in the shape of a rotary knob and/or a keypad or the like for inputting data into the respective infusion device 2, for example for programming an infusion operation, and a display for outputting information, for example relating to an ongoing infusion operation.

The following figures show an embodiment of the portable rack 20 according to the present invention. In contrast to the rack 10 described in FIG. 1, the portable rack 20 is not serving as a communication link but as a power supply only. The power supply is provided such that the infusion devices 2 connected to the portable rack 20 are connected to a power supply via the portable rack 20. The portable rack 20 is protected against short circuits with the modules 2 it holds.

The portable rack 20 is intended to act as a support mechanism and centralized power supply for two infusion modules 2, in particular when two modules 2 are running on battery and need to be transported. FIG. 2a shows a front view of the portable rack 20 without any attached infusion device 2. FIG. 2b shows the back view of the portable rack 20.

FIG. 3 shows that two modules 2 are mounted on the portable rack 20. The portable rack 20 is designed to hold and power two modules 2: a volumetric pump or a syringe pump. The upper module 2 is a volumetric pump. The lower module 2 is a syringe pump. With this portable rack 20, the user can carry the modules 2 with the handle 22 or can attach the modules 2 to a pole or rail. For instance, the portable rack 20 can be installed on a pole, rail or bed-rail with a diameter between 16 mm and 40 mm (not shown in the figures).

FIGS. 4 and 5 illustrate installing the portable rack 20 and the modules 2.

At first, FIG. 4 illustrates installing the portable rack 20:
1. To attach the portable rack 20 directly to the pole: push down on the release button 28, slide the screw clamp 27 forward and tighten it. The portable rack 20 can also be attached to a horizontal rail. To do so, the pole clamp 27 must be repositioned vertically. The portable rack 20 can also be placed directly onto a flat surface, such as a tablet, table, cupboard, shelf, nightstand or the patients bed (but not on the floor). To do so, the pole clamp 27 must or should be removed.
2. Plug the portable rack 20 into the power supply 29 via a cable (not shown in the figures). The wall socket must remain accessible at all times to allow emergency power supply disconnection.

The technique of the screw clamp 27 bases on a technique or is at least similar to a technique which is for instance described in the patent application DE 10 2015 122 699 A1. The entire content of this patent application is incorporated by reference.

FIG. 5 now illustrates installing the modules 2 to the portable rack 20.

With the hands on each side of the module 2, slide the module into the portable rack 20, making sure that you hear it click into place. It is recommended inserting the device 2 into the bottom slot first. The device 2 turns on when it is plugged to the mains power supply. There is no ON/OFF button. Unplug the device to turn it off. The portable rack 20 can connect to and power two modules 2. A correct connection is signaled by an audible click. The rack cannot be configured.

FIGS. 6 to 8 illustrate removal and transport of the portable rack 20 and the modules 2.

At first, FIG. 6 illustrates the removal of the modules 2 from the infusion station IS, for instance when moving the module 2 with the patient P. Before transporting the patient P, make sure the modules battery is sufficiently charged. First, remove the module 2 by pressing the blue release button on the back-right corner, and slide the module 2 out of the rack of the infusion station IS. When the module 2 is on, the touchscreen and keypad automatically lock when you press the release button. The module 2 beeps when it switches to battery power supply.

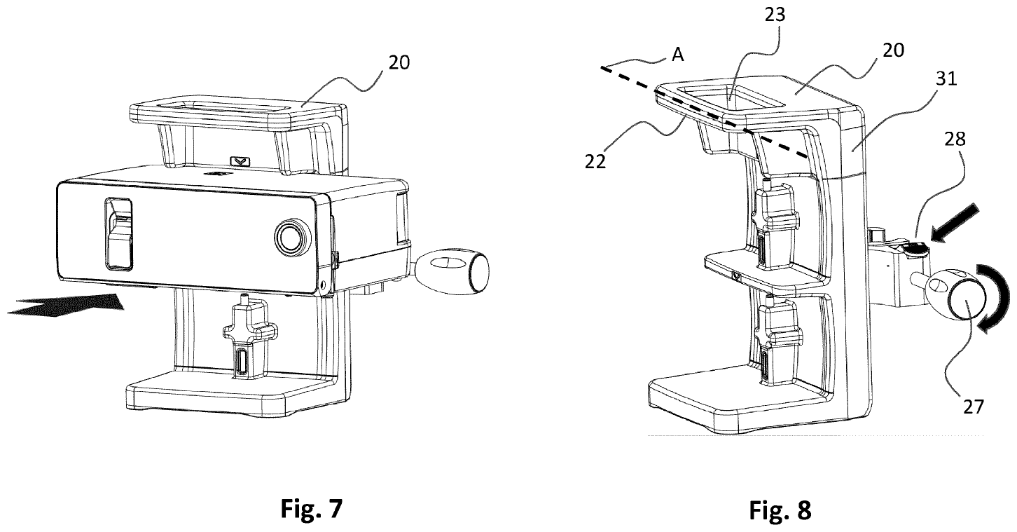

Install the module 2 in the portable rack 20. With your hands on each side of the module 2, slide the module 2 into the portable rack 20, making sure that you hear it click into place (see FIG. 7). Connect the portable rack 20 to the mains once the patient P has arrived at the destination, and once the patient P is transferred back to bed, re-install the module 2 in the infusion station IS. The module 2 beeps when it switches to the mains power supply. The power status LED lights up green and the battery charge status LED blinks to indicate that the battery is charging.

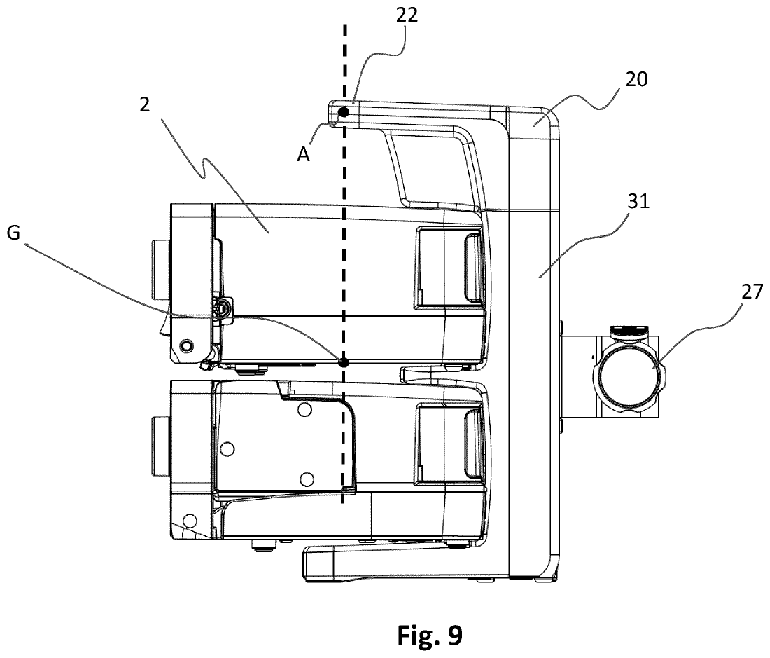

Disassembling the portable rack 20 is illustrated in FIG. 8. 1. Remove all the modules 2 from the portable rack 20. 2. Push down on the release button 28 and pull on the screw clamp 28 to release it from the pole or rail.

FIG. 8 also depicts the position of the handle 22 extending along an axis A following a transversal direction and defining, in the upper side of the portable rack 20, the opening 23, allowing for a user to manipulate and to transport the portable rack 20.

As it can be further contemplated in FIG. 9, the position of the handle 22 relative to the back 31 of the portable rack 20 is such that, when one or several infusion devices 2—for instance two infusion devices 2—are loaded onto the portable racks slot 100, the back 31 of the portable rack 20 stands essentially vertically. Essential vertical means that portable rack 20 could be slightly tilted. This is achieved by the following features:
the center of gravity (G) of the infusion devices 2 being located in the region of the center of the infusion device preferably essentially at the center of the infusion device 2,
the axis A of the handle 22 being located such that it aligns with the middle of the infusion devices 2 when loaded, and
the negligible weight of the portable rack in regard to the weight of the infusion devices 20.

As a result, in particular during manual transportation of the loaded portable rack 2, the infusion devices 2 stand horizontally such that their screens can be seen.

It will be understood that the invention may be embodied in other specific forms without departing from the spirit or central characteristics thereof. The present examples and embodiments, therefore, are to be considered in all respects as illustrative and not restrictive, and the invention is not to be limited to the details given herein. Accordingly, features of the above described specific embodiments can be combined with one another. Further, features described in the summary of the invention can be combined with one another. Furthermore, features of the above described specific embodiments and features described in the summary of the invention can be combined with one another.

### LIST OF REFERENCE NUMERALS

A Axis (of the handle)
G Center of gravity (of the infusion devices)
IS Infusion station
P Patient
2 Infusion device/module
3 Stand
10 rack
100 Slot
11 Control device
12 Interface device
20 Portable rack
21 Housing
22 Handle
23 Opening or aperture (in the housing)
24 Base (of the housing)
25 Connector for power supply
26 Mounting structure for the infusion pump
27 Clamp
28 Release button
29 Power inlet
30 Device identification label
31 Back (of the portable rack)

1. A portable rack for holding infusion devices for administering a medical fluid to a patient, comprising:
a housing with a multiplicity of slots wherein the infusion devices can be attached to said multiplicity of slots,
a clamp for mounting the rack to a pole and/or a rail, and
a handle for manually transporting the rack.

2. The portable rack according to claim 1, wherein the handle is arranged on a top side of the rack and is preferably integral with the housing of the rack.

3. The portable rack according to claim 1, wherein the handle is provided by an opening in a top side of the housing.

4. The portable rack according to claim 1, wherein the handle is provided in a front part of a top side of the housing.

5. The portable rack according to claim 1, wherein the clamp is a screw pole clamp.

6. The portable rack according to claim 5, wherein the clamp has a release mechanism for sliding the screw pole clamp forward and for tighten it.

7. The portable rack according to claim 1, wherein the clamp can be positioned vertically and horizontally onto the pole and/or the rail.

8. The portable rack according to claim 1, wherein the rack can be placed directly onto an essentially horizontal and/or preferably flat surface.

9. The portable rack according to claim 1, further comprising a flat base.

10. The portable rack according to claim 9, wherein the base comprises rubber feet.

11. The portable rack according to claim 1, further comprising a power supply for the infusion devices and a communication link for the infusion devices.

12. The portable rack according to claim 11, wherein the power supply is for the infusion devices only.

13. The portable rack according to claim 1, wherein the housing has two slots for attaching two infusion devices only.

14. The portable rack according to claim 1, wherein the handle is positioned such that, when at least one infusion device is loaded onto a slot, a back of the portable rack stands essentially vertically during manual transportation of the portable rack.
